(19) United States
(12) Patent Application Publication
(10) Pub. No.: US 2016/0338712 A1
(43) Pub. Date: Nov. 24, 2016

(54) Punch Instrument

(71) Applicant: ZIMMER, INC., Warsaw, IN (US)

(72) Inventors: John Chernosky, Brick, NJ (US); Ray Zubok, Norht Haledon, NJ (US); Timothy A. Hoeman, Morris Plains, NJ (US); Imants Liepins, Asbury, NJ (US); Keith A. Roby, Jersey City, NJ (US); Greg Stebbins, Hobeken, NJ (US); Sara Savoia, Edison, NJ (US)

(21) Appl. No.: 15/111,505

(22) PCT Filed: Jan. 20, 2015
(86) PCT No.: PCT/US2015/011954

§ 371 Date: Jul. 14, 2016

Related U.S. Application Data
(60) Provisional application No. 61/931,092, filed on Jan. 24, 2014.

(51) Int. Cl. A61B 17/16

(52) U.S. Cl. CPC A61B 17/1604; A61B 17/1675

(57) ABSTRACT

Punch instruments (5) are provided in some embodiments for cutting tissue in a resected proximal tibia. Such instruments can include an elongate handle (10) and a cutting head (20) disposed at the distal end of the elongate handle (10). The cutting head (20) can include a first annular blade member (25) with a first leading cutting edge (29) that extends generally transversely to a longitudinal axis of the elongate handle (10). The cutting head (20) can include a second annular blade member (26) that is situated inwardly of the first annular blade member (25) and which includes a second leading cutting edge (30) that extends generally transversely to the longitudinal axis of the elongate handle (10).

### CLAIM OF PRIORITY

This application claims the benefit of U.S. Provisional Patent Application Ser. No. 61/931,092, filed on Jan. 24, 2014, the benefit of priority of which is claimed hereby, and which is incorporated by reference herein in its entirety.

### TECHNICAL FIELD

This document pertains generally, but not by way of limitation, to tibial punches for bone preparation.

### BACKGROUND

In many surgical procedures involving prosthetic implants, a space or void in a bone can be created that can receive an implant component. The bone material can include a hard outer cortical bone material and a softer inner cancellous bone material. In many instances, it may be important not to damage the cortical bone because the cortical bone provides structural strength to the bone. Prior methods of creating a space in a bone include broaching and cutting with a burr. These methods can damage the cortical bone.

### OVERVIEW

The present inventors recognize, among other things, that a safe bone punch can be beneficial. The present disclosure provides, in certain aspects, an orthopedic device that can create a space in a bone without damage to a cortical bone. For example, a punch can be provided with a wide array of cutting head configurations used to create a space or void that can receive a prosthesis component. The punch can include a cutting head having at least two blades. The blades can be shaped such that incisions in the bone can act as a template or guide to aid a surgeon in resecting a desired void shape. For example, to create a cone shaped void, the cutting head can have a series of blades with an outermost blade penetrating a bone to a shallow depth, while an innermost blade can penetrate a bone to a deepest depth. After these incisions have been made, a surgeon can use the incisions as a guide to remove bone and create the desired void shape.

To further illustrate the punch instrument and method disclosed herein, a non-limiting list of examples is provided here:

In Example 1, a punch for cutting tissue in a resected proximal tibia, can comprise an elongate handle having a longitudinal axis and include a proximal end and a distal end; and a cutting head disposed at the distal end of the elongate handle. The cutting head can include a first annular blade member with a first leading cutting edge that extends generally transversely to the longitudinal axis of the elongate handle, and wherein the cutting head can also include a second annular blade member that is situated inwardly of the first annular blade member and which includes a second leading cutting edge that extends generally transversely to the longitudinal axis of the elongate handle.

In Example 2, the punch of Example 1 can optionally be configured such that at least one of the first leading cutting edge and the second leading cutting edge extends in multiple planes.

In Example 3, the punch of any one or any combination of Examples 1 or 2 can optionally be configured such that the first leading cutting edge extends in a first plane.

In Example 4, the punch of Example 3 can optionally be configured such that the second leading cutting edge extends in a second plane that is parallel with the first plane.

In Example 5, the punch of Example 3 can optionally be configured such that the second leading cutting edge extends in a second plane that is non-parallel with the first plane.

In Example 6, the punch of any one or any combination of Examples 1-5 can optionally be configured such that the second leading cutting edge extends distally of the first leading cutting edge.

In Example 7, the punch of any one or any combination of Examples 1-6 can optionally be configured such that the first leading cutting edge and the second leading cutting edge have the same perimeter shape.

In Example 8, the punch of any one or any combination of Examples 1-7 can optionally be configured such that at least one of the first leading cutting edge and the second leading cutting edge is curvilinear.

In Example 9, the punch of any one or any combination of Examples 1-8 can optionally be configured such that at least one of the first annular blade member and the second annular blade member includes a cylindrical longitudinal section.

In Example 10, the punch of any one or any combination of Examples 1-9 can optionally be configured such that at least one of the first annular blade member and the second annular blade member includes a conical longitudinal section.

In Example 11, the punch of any one or any combination of Examples 1-10 can optionally be configured to further comprise a backing plate from which at least one of the first annular blade member and the second annular blade member extends.

In Example 12, the punch of any one or any combination of Examples 1-11 can optionally be configured such that the cutting head further includes a first linear blade member that is surrounded by the second annular blade member and which includes a first linear cutting edge that extends generally transversely to the longitudinal axis of the elongate handle.

In Example 13, a punch for cutting tissue in a resected proximal tibia, can comprise an elongate handle that includes a proximal end and a distal end and a cutting head disposed at the distal end of the elongate handle. The cutting head can include a first annular blade member into which cut tissue surrounded by the first annular blade member can pass when the punch is impacted and a second annular blade member that is generally coaxial with the first annular blade member and into which cut tissue surrounded by the second annular blade member can pass when the punch is impacted into the resected proximal tibia. The first annular blade member can include a first leading cutting edge, and the second annular blade member can include a second leading cutting edge that encompasses a smaller area than the first leading cutting edge and that extends distally of the first leading cutting edge.

In Example 14, the punch of Example 13 can optionally be configured such that the first annular blade member and the second annular blade member are modular components separately or concurrently connectable to the elongate handle.

In Example 15, the punch of any one or any combination of Examples 13 or 14 can optionally be configured such that at least one of the first annular blade member and the second annular blade member extends in perpendicular manner from a backing plate situated at the distal end of the elongate handle.

In Example 16, a punch for cutting tissue in a resected proximal tibia, can comprise an elongate handle that includes a proximal end and a distal end and a cutting head disposed at the distal end of the elongate handle. The cutting head can include a first annular cutting edge for making a first circumferential cut in the resected proximal tibia, and wherein the cutting head can include a second annular cutting edge for making a second circumferential cut in the resected proximal tibia that is encompassed by the first circumferential cut.

In Example 17, the punch of Example 16 can optionally be configured such that the entirety of said second annular cutting edge extends further from the elongate handle than the entirety of the first annular cutting edge for cutting deeper into the resected proximal tibia than the first annular cutting edge.

In Example 18, the punch of any one or any combination of Examples 16 or 17 can optionally be configured such that at least one of said first circumferential cut and said second circumferential cut are coaxial.

In Example 19, the punch of any one or any combination of Examples 16-18 can optionally be configured such that the first annular cutting edge extends in a first plane, and wherein said second annular cutting edge extends in a second plane that is parallel with said first plane.

In Example 20, a method for cutting tissue in a resected proximal tibia, can comprise providing a tibia punch that includes an outer annular blade member that is generally coaxial with an inner annular blade member, the inner annular blade member encompassing a smaller cutting area than said outer annular blade member and extending distally of said outer annular blade member. The method can also comprise forcing the tibia punch into a resected proximal tibia, wherein the inner annular blade member enters the resected proximal tibia before the outer annular blade member, with an inner cut tissue surrounded by the inner annular blade member passing into the inner annular blade member and an outer cut tissue surrounded by the outer annular blade member passing into the outer annular blade member.

In Example 21, the method of Example 20 can optionally be configured to further comprise removing the tibia punch from the resected proximal tibia, wherein the inner cut tissue passes back through the inner annular blade member and the outer cut tissue passes back through the outer annular blade member, with the inner cut tissue and the outer cut tissue each including tissue portions still attached to the resected proximal tibia.

In Example 22, the method of Example 21 can optionally be configured to further comprise removing the inner cut tissue and the outer cut tissue from the resected proximal tibia which includes detaching the tissue portions from the resected proximal tibia.

In Example 23, the punch and method of any one or any combination of Examples 1-22 can optionally be configured such that all elements, operations, or other options recited are available to use or select from.

Each of these non-limiting examples can stand on its own, or can be combined in various permutations or combinations with one or more of the other examples.

### BRIEF DESCRIPTION OF THE DRAWINGS

In the drawings, like numerals can be used to describe similar elements throughout the several views. Like numerals can be used to represent different views or configurations of similar elements. The drawings illustrate generally, by way of example, but not by way of limitation, various embodiments discussed in the present document.

FIG. 1 illustrates an isometric view of a punch instrument as viewed from a side, as constructed in accordance with at least one embodiment.

FIG. 2 illustrates an isometric view of a punch instrument as viewed from the top, as constructed in accordance with at least one embodiment.

FIG. 3 illustrates an isometric view of a punch instrument as viewed from the bottom, as constructed in accordance with at least one embodiment.

FIG. 4 illustrates a top view of a resected bone with incisions made by a punch instrument.

FIG. 5A illustrates an isometric view of a cutting head of a punch instrument, as constructed in accordance with at least one embodiment.

FIG. 5B illustrates an isometric view of a cutting head of a punch instrument, as constructed in accordance with at least one embodiment.

FIG. 6 illustrates an isometric view of a rim-less cutting head of a punch instrument, as constructed in accordance with at least one embodiment.

FIG. 7 illustrates an isometric view of a rimmed cutting head of a punch instrument, as constructed in accordance with at least one embodiment.

FIG. 8 illustrates an isometric view of a cutting head of a punch instrument, as constructed in accordance with at least one embodiment.

FIG. 9 illustrates an isometric view of a cutting head of a punch instrument, as constructed in accordance with at least one embodiment.

FIG. 10 illustrates an isometric view of a punch instrument as viewed from the bottom, as constructed in accordance with at least one embodiment.

FIG. 11 illustrates an isometric view of a cutting head of a punch instrument, as constructed in accordance with at least one embodiment.

FIG. 12 illustrates a side view of a cutting head of a punch instrument, as constructed in accordance with at least one embodiment.

FIG. 13 illustrates an isometric view of a conical cutting blade member as viewed from the top, as constructed in accordance with at least one embodiment.

FIG. 14 illustrates an isometric view of a conical cutting blade member as viewed from a side, as constructed in accordance with at least one embodiment.

### DETAILED DESCRIPTION

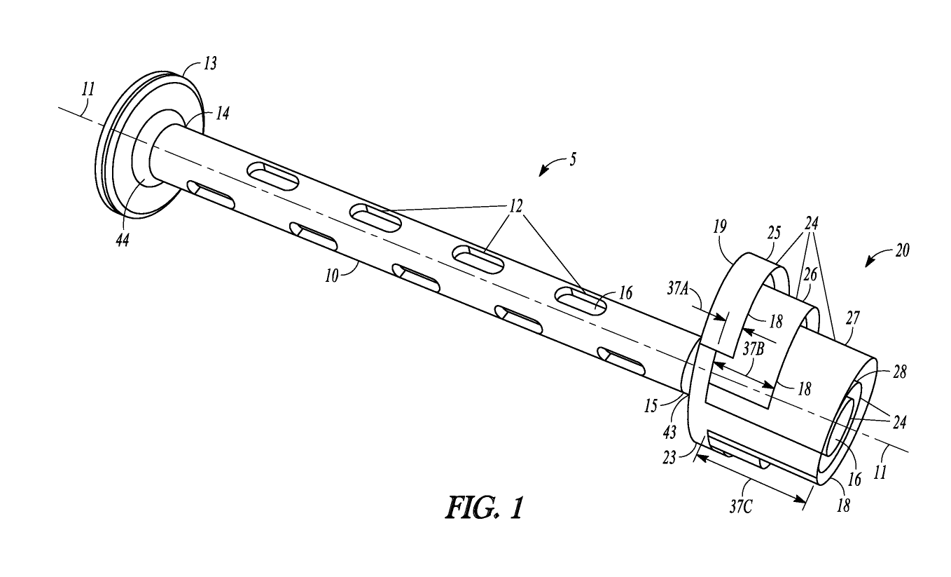

FIGS. 1, 2, and 3 illustrate an example of a punch instrument 5 from side, top and bottom views respectively. The punch 5 can include a handle 10 extending along a longitudinal axis 11 from a proximal end 14 to a distal end 15. A cutting head 20 can be located at the distal end of the handle 10. An impact member 13 can be located at the proximal end 14 of the handle 10. The punch 5 can be impacted into a bone at a surgical site and because the cutting head 20 can include two or more cutting blade members 24, the punch 5 can create a series of incisions in a bone site. The impact member 13 is shown in FIG. 1 as circular, but can be any shape configured to receive blows from an impact tool such as a hammer or blows from an open hand. The impact member 13 can be integral or connectable to the handle 10. A first connection 44 between the handle 10 and the impact member 13 can include welding, brazing, threading, a taper fit, an interference fit, a quick release connection or any other means to securely connect the handle 10 to the impact member 13. In an example, the punch 5 can be used without an impact member 13. The handle 10 can be round, oval, rectangular, or shaped to aid gripping by hand. The handle 10 can be solid or hollow.

The punch 5 can include a central bore or cannulation 16 extending through the impact member 13, the handle 10, and the cutting head 20. A cannulation 16 can allow the punch 5 to be slid over an intramedullary rod positioned in a surgical site. The intramedullary rod can aid in the placement of the punch 5 for creating a space in a bone. The handle 10 can include one or more handle apertures 12 which can pass from the surface of the handle 10 through to the cannulation 16. If the handle 10 is not cannulated, the handle apertures 12 can pass through the handle 10. The handle apertures 12 can be used for cleaning the punch 5 or for visibility of an intramedullary rod positioned within the cannulation 16.

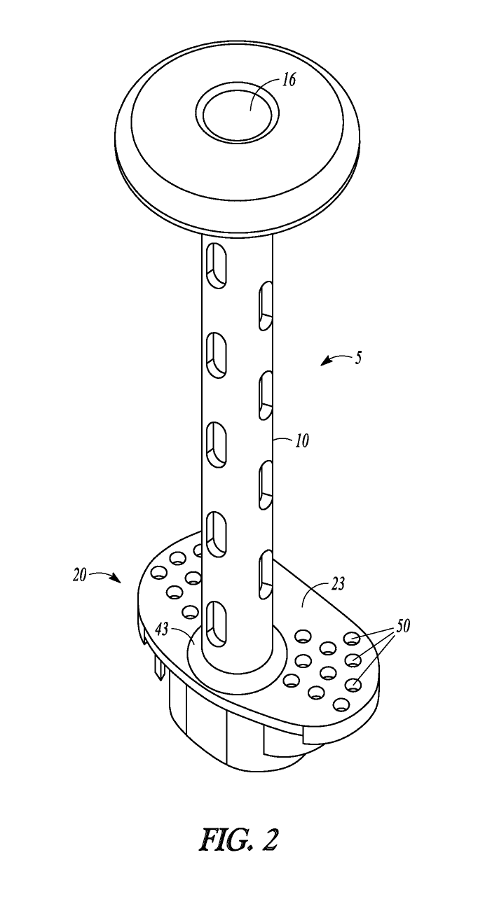

The cutting head 20 can be configured with cutting blade members 24 in a wide variety of embodiments to provide incisions in a bone site to aid in producing a cavity for a prosthetic component. The cutting blade members 24 can be connected at a cutting head proximal end 19 to a backing plate 23. When present, the backing plate 23 can be generally transverse to the longitudinal axis 11. Cutting blade members 24 can be connected to each other or to the handle 10 by means other than a backing plate 23, such as by supporting members (not illustrated). The supporting members can be a series of spokes or tubular members. Each cutting blade member 24 can extend from a cutting head proximal end 19 to a leading cutting edge 18. The cutting blade member 24 can be shaped in a variety of configurations as seen in FIGS. 1-3 and 5-14. As shown in FIG. 2, the cutting head 20, can be integrally formed with the handle 10 or can be attached at a second connection 43. A second connection 43 between the handle 10 and the cutting head 20 can include welding, brazing, threading, a taper fit, an interference fit, a quick release connection or any other means to securely connect the handle 10 to the cutting head 20. The cutting head 20 can be removable so that a series of differently configured cutting heads 20 can be removed and installed on a handle 10. A backing plate 23 can include one or more cutting head apertures 50. These apertures can provide visual aids for the operator of the punch 5 such as an indication of when the backing plate 23 is resting on a bone surface. The cutting head apertures 50 can also provide access for cleaning purposes or debris removal from the cutting head 20. The punch 5 and its components: the handle 10, the impact member 13, the cutting head 20, and the cutting blade members 24 can be manufactured of metals, metal alloys, plastics, ceramics and combinations thereof.

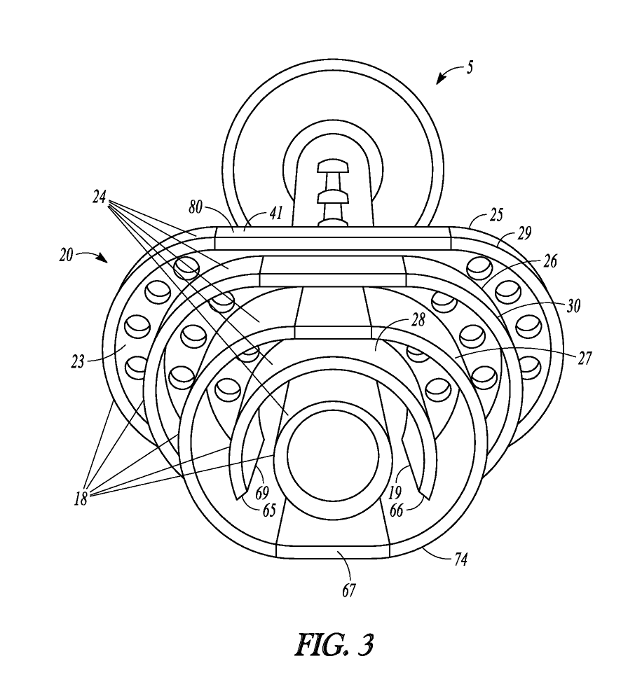

In FIG. 3, a punch 5 is depicted that includes a cutting head 20 having five cutting blade members 24. A first annular blade member 25 can be an outermost blade member and can form a perimeter shape 41 that can also be the perimeter shape 41 of the backing plate 23. A second annular blade member 26 can be positioned inside of the first annular blade member 25. Additional blade members such as a third annular blade member 27 and a fourth annular blade member 28 can also be located on the cutting head 20. As shown in FIG. 1, the cutting blade members can extend to differing lengths from the cutting head proximal end 19. The first annular blade member 25 can extend the shortest distance from the cutting head proximal end 19 and as cutting blade members 24 are positioned closer to the longitudinal axis 11 the length of the cutting blade member 24 can increase. The cutting blade members 24 can have lengths that are generally the same, such as the third annular blade member 27 and fourth annular blade member 28 shown in FIG. 1. The length of each cutting blade member 24 can control a cutting depth 37A, 37B, and 37C. The cutting depths 37A, 37B, and 37C can be a distance from a leading cutting edge 18 to the backing plate 23.

FIG. 3 illustrates a third annular cutting blade member 27 that is a continuous loop 67 wherein a third leading cutting edge 74 has no openings or non-cutting portions. A fourth annular cutting blade member 28 is not a continuous loop 67 and can include a first end 65 and a second end 66 which are not connected along a vertical edge 69 extending from a leading cutting edge 18 to the proximal end 19 of the fourth annular cutting blade member 28. Any cutting blade member 24 can be configured as either a continuous loop 67 or non-continuous as the fourth annular cutting blade member 28. Each cutting blade member 24 can be sharpened at the leading cutting edge 18. The cutting edge can be configured as beveled on one side, beveled on both sides or in any other known manner of providing a sharpened edge. The leading cutting edge 18 of each cutting blade member 24 can form a plane or be serrated or undulating. The first annular blade member 25 can include a first leading cutting edge 29. The second annular blade member 26 can include a second leading cutting edge 30.

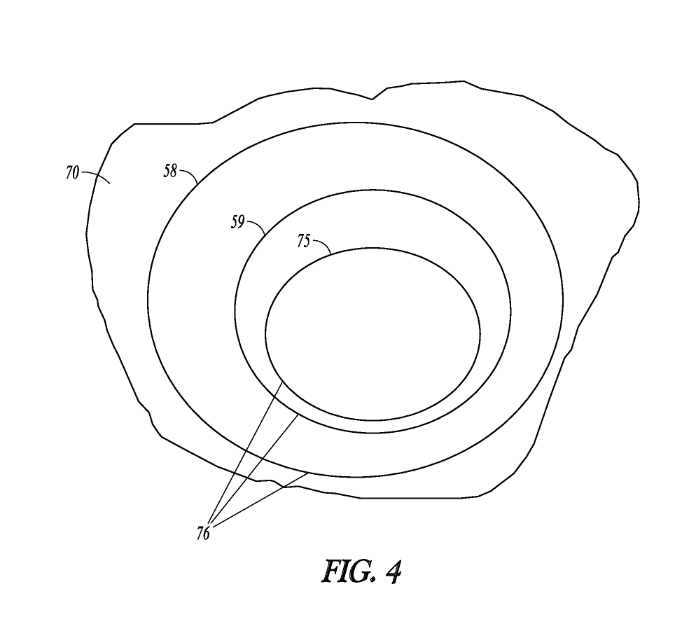

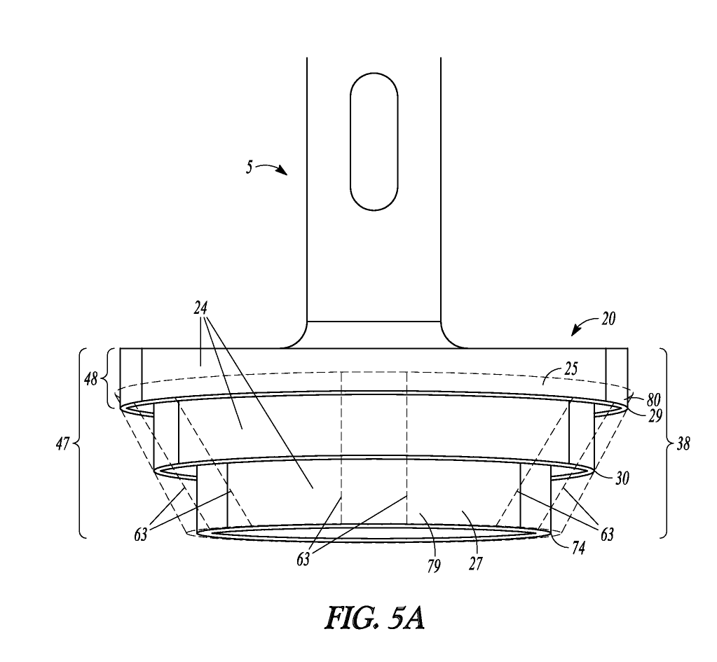

FIG. 4 illustrates a top view of a resected tibial bone 70. When the punch 5 is impacted at a surgical site, the first leading cutting edge 29 (see FIG. 5A) can create a first circumferential cut 58 in a resected bone 70. A second leading cutting edge 30 (see FIG. 5A) can create a second circumferential cut 59. A third leading cutting edge 74 (see FIG. 5A) can create a third circumferential cut 75. Each respective blade can create its own cut in the bone. For example, the punch 5 (see FIG. 5A) can be impacted into the surface of a resected tibial bone. The cutting blade members 24 make cuts or incisions 76 in the bone 70 and these incisions can be used as a general guide or a template for removing bone outlined by the incisions. In practice, an innermost blade member 79 (see FIG. 5A) can be longer than outer blades and will contact the bone surface first and cut to the greatest depth. As the punch 5 is driven deeper into a bone site, an outermost blade member 80 (see FIG. 5A) will contact the bone 70 last and make the shallowest incision 76 in the bone 70. The blades in a cutting head can be configured to provide an approximation for any three-dimensionally shaped void required for an installation of a prosthetic component. In some instances, incisions with a cutting head will create an approximate shape, and then the surgeon will fine tune the shape by hand with a scalpel or other separate cutting instrument, for example, by cutting along lines connecting the various incisions. FIG. 5A illustrates a cutting head 20 of a punch 5. The cutting blade members 24 can form a stepped configuration 38 whereby an innermost blade member 79, such as a third annular cutting blade member 27 has the greatest cutting depth 47 and an outermost blade member 80, such as a first annular cutting blade member 25 can have the least amount of cutting depth 48. Bone removal lines 63 show locations where a surgeon can interconnect cuts made by the three cutting blade members 24 pictured to create a void in a bone site shaped to receive a prosthetic component.

FIG. 5B illustrates a cutting head 20 of a punch 5. The cutting blade members 24 can form a stepped configuration 38 whereby an innermost blade member 79, such as a third annular cutting blade member 27 has the greatest cutting depth 47 and an outermost blade member 80, such as a first annular cutting blade member 25 can have the least amount of cutting depth 48. While merely illustrative, bone removal lines 63 show locations where a surgeon can make additional cuts around one or more of the blade incisions. A surgeon can use any guidelines created by the cutting blade members 24, such as a first leading cutting edge 29, or an angle A created by a third leading cutting edge 74 to aid in creating a void in the bone site. Angle A can be formed between a transverse plane 9, which is generally transverse to the longitudinal axis 11 and a plane formed by the third leading cutting edge 74. Angle A can be formed between the transverse plane 9 and any leading cutting edge 18. Angle A can be any angle from 0-85 degrees. In an example, any leading cutting edge 18 can be configured such that angle A can be any angle from 0-85 degrees. In an example, any leading cutting edge 18 can be configured such that angle A can be any angle from 0-45 degrees. In an example, any leading cutting edge 18 can be configured such that angle A can be any angle from 0-30 degrees. In an example, any leading cutting edge 18 can be configured such that angle A can be any angle from 0-15 degrees. In an example, any leading cutting edge 18 can be configured such that angle A can be any angle from 0-10 degrees. In an example, any leading cutting edge 18 can be configured such that angle A can be any angle from 0-5 degrees. A first blade section 91 of the second annular blade member 26 or a second blade section 92 of a third annular blade member 27 can cut deeper into a cancellous bone than bone removal lines 63 created by a surgeon to create a void in a bone site.

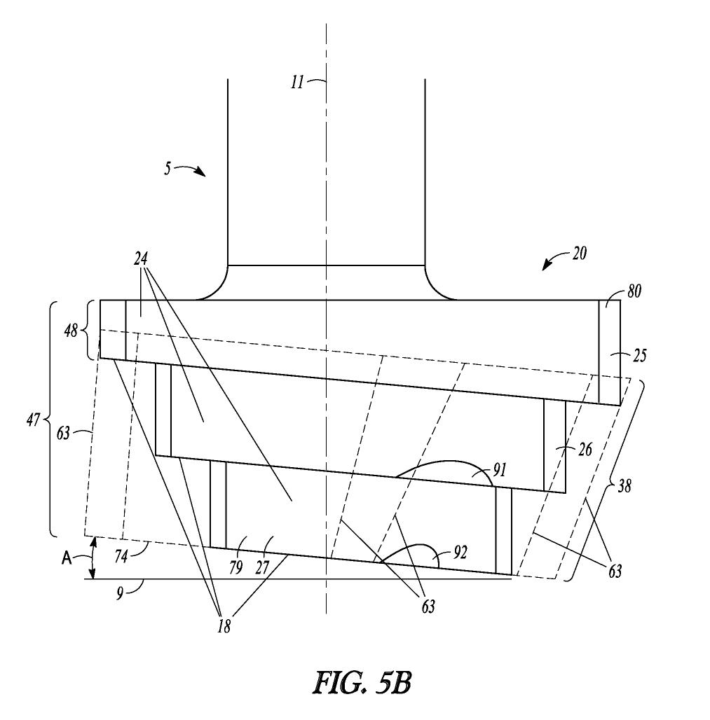

FIG. 6 illustrates another configuration for a cutting head 20 that includes two cutting blade members 24. The first annular blade member 25 can follow the perimeter shape 41 of the backing plate 23. The first annular blade member 25 can include a first leading cutting edge 29 which can produce a first circumferential cut 58 in a bone site (See FIG. 4). The second annular blade member 26 can have a different shape than the first annular blade member 25. At a lobe 71, the second annular blade member 26 can be exterior to the perimeter shape 41 of the first annular blade member 25 or the backing plate 23.

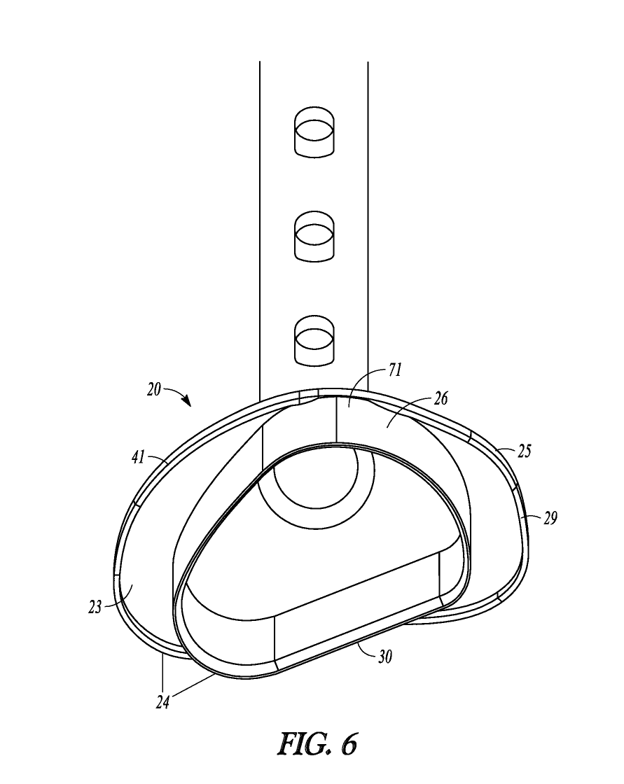

FIG. 7 illustrates a cutting head 20 that is almost identical to the cutting head 20 illustrated in FIG. 6, except that the backing plate 23 has a rim 21 extending in a direction outwardly from the longitudinal axis 11 and from the outer surfaces of the first cutting blade member 25. The rim 21 can be shaped to provide location guidance when compared to the resected tibial bone and can also provide additional contact areas to outer cortical bone as the punch 5 is driven to a maximum depth into cancellous bone. Any configuration of cutting head 20 can be provided with a rim 21 and the rim 21 can be shaped in a wide variety of configurations.

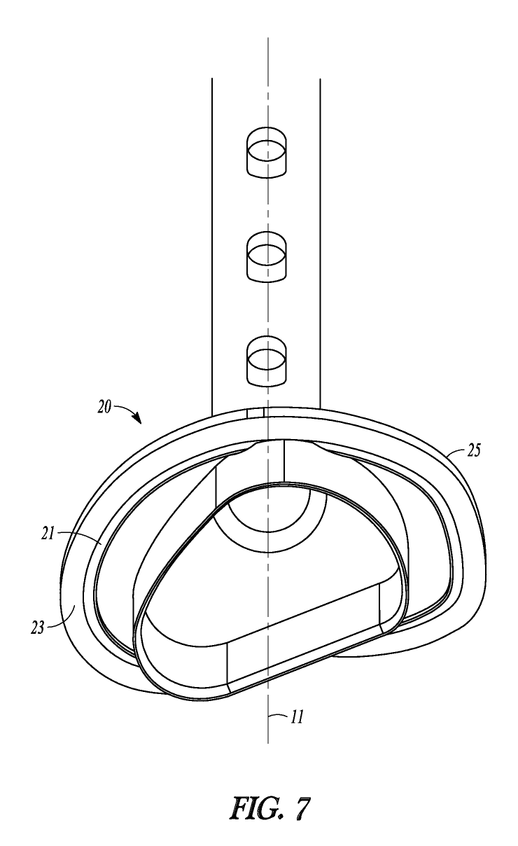

FIG. 8 illustrates a cutting head 20 having a scallop shaped rim 52. The scallop shaped rim 52 can include one or more cut outs 56. The cut outs 56 can extend to the outer edge 60 of the first annular blade member 25 such as at location 57. The scalloped shaped rim 52 can allow a surgeon to visualize the outer perimeter of the first annular blade member 25 through the access provided by the cut outs 56. A backing plate 23 can include cutting head apertures 50 that define large irregular shapes or small circular shapes as in FIG. 2. The cutting head apertures 50 can be used to aid visibility of placement of the cutting blade members 24 or a depth of the cutting head during use of the punch 5 and can benefit cleaning of the punch 5.

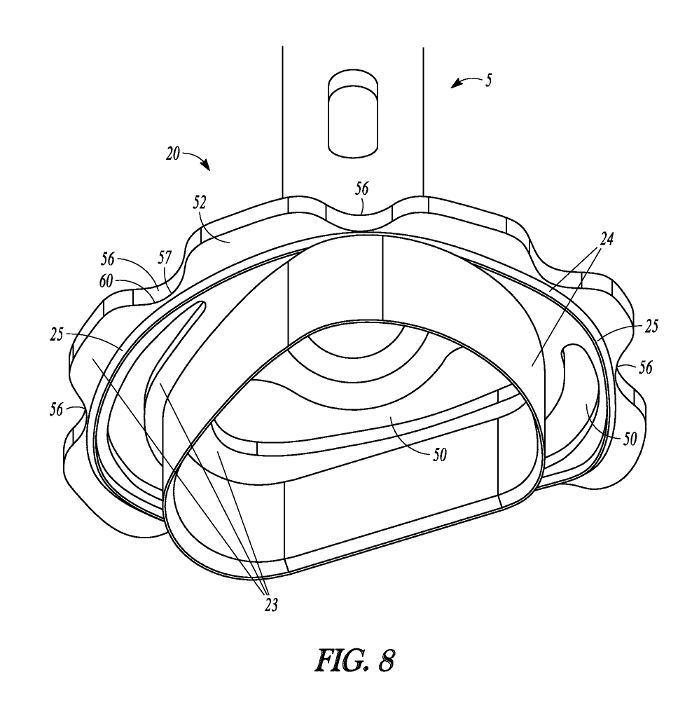

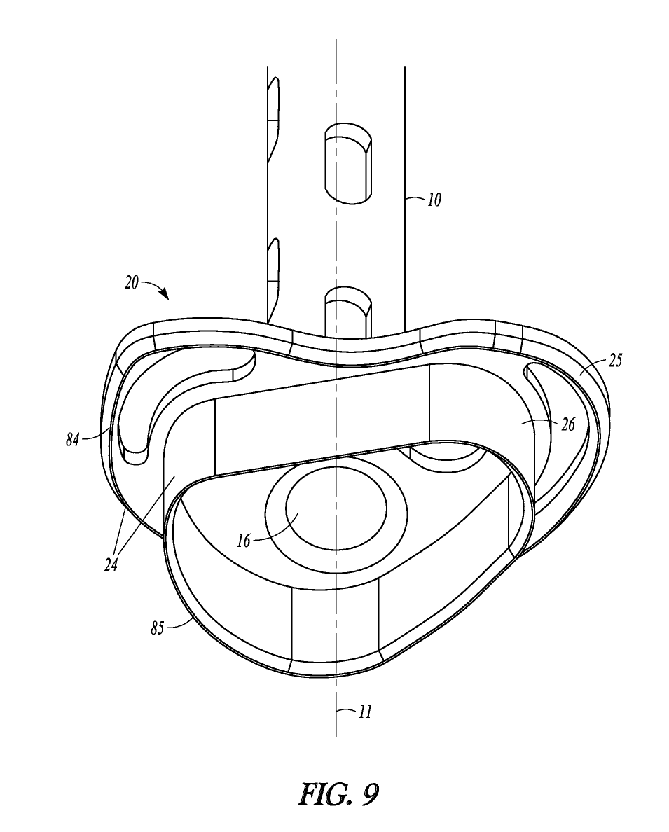

FIG. 9 illustrates another configuration of a cutting head 20. A second annular blade member 26 can have a second perimeter shape 85 that diverges or is quite different from a first perimeter shape 84 of a first annular blade member 25. The distal opening of the cannulation 16 can be congruous with the long axis 11 of the handle 10. In FIG. 10, a punch 5 is illustrated with a cutting head 20. The cutting head 20 can have cutting blade members 24 having similar perimeter shapes 41. The first annular cutting blade member 25 can be an outermost blade member 80. A second annular cutting blade member 26 can be positioned inside the first annular cutting blade member 25 and a third annular cutting blade member 27 can be positioned inside the second annular cutting blade member 26. The third annular cutting blade member 27 can be an innermost blade member 79.

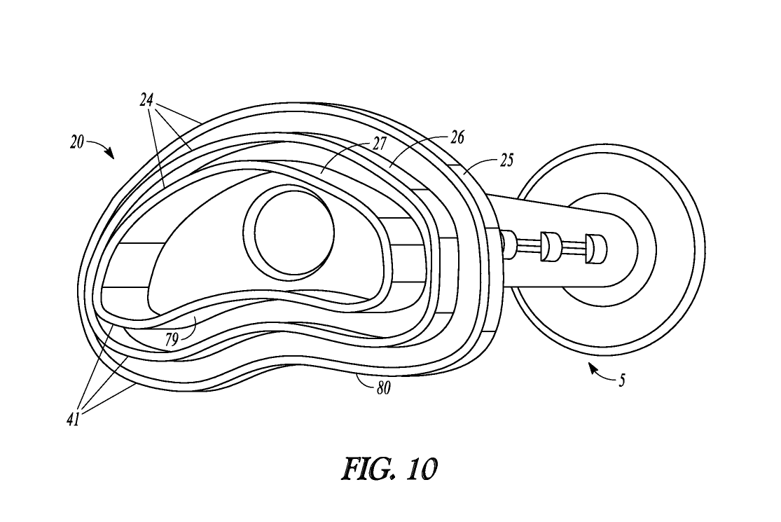

FIG. 11 illustrates another example of a cutting head 20. The cutting blade members 24 can have a curvilinear shape 53. Such a curvilinear shape 53 can include one or more positive radius portions 95 and one or more negative radius portions 96. A positive radius portion 95 curves outwardly away from a longitudinal axis 11 and a negative radius portion 96 curves inwardly towards a longitudinal axis 11. Such a curvilinear shape 53 of a second cutting blade member 26 can include a small positive radius portion 87, a large positive radius portion 88, a generally straight portion 90, and a negative radius portion 89. The shape of each cutting blade member 24 can have any combination of any size or direction of radii, straight portions, symmetricalness, or assymmetricalness in sizes or dimensions necessary in order to produce a required incision. The cutting head 20 can include one or more straight shaped blade members 71. The straight shaped blades members 71 are illustrated as located inside of a third annular cutting blade member 27. In other examples, straight shaped blades 71 can be located anywhere in the cutting head 20, for example between a first annular blade member 25 and a second annular blade member 26. In another example one or more straight shaped blade members 71 can be located between a second annular blade member 26 and a third annular blade member 27. The straight shaped blades 71 can be located in all or any combinations of these areas of the cutting head 20 and aligned in any direction. The cutting edge of the straight shaped blade 71 can have an angle. For example if a straight shaped cutting blade 71 is positioned between a first annular blade member 25 and a second annular blade member 26 with the blade generally transverse to the general direction of the curvilinear shape 53, the edge closest to the a first annular blade member 25 can have a cutting depth substantially equal to the cutting depth of the first annular blade member 25. In addition, the edge of the straight shaped cutting blade 71 that is closest to the second annular blade member 26 can have a cutting depth substantially equal to the cutting depth of the second annular blade member 26.

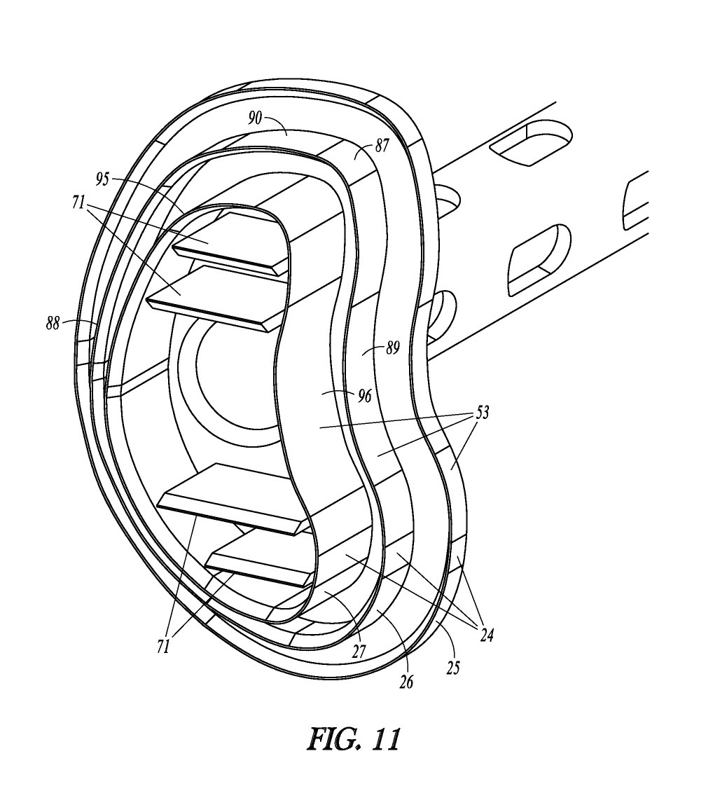

FIG. 12 illustrates a cutting head 20 having three cutting blade members 24 in a stepped configuration 38. Such a stepped configuration 38 has a series of cutting blade members 24 that cut to increasingly deeper depths moving from an outermost blade member 80 to an innermost blade member 79. The first leading cutting edge 29 can form a first plane 39 if all points along the first leading cutting edge 29 are generally in the same plane. Similarly, the second leading cutting edge 30 can form a second plane 40. The angular relationship between the first plane 39 and the second plane 40 can be varied from one cutting head configuration to another. In an example, the cutting edge planes of the cutting head 20 can be parallel to a plane transverse 82 (hereafter referred to as transverse plane 82) to the longitudinal axis 11. In another example an angle A between the transverse plane 82 to the longitudinal axis 11 of the handle 10 and a first cutting edge plane 39 can be an angle from 0-45 degrees. In another example an angle B between the plane of a backing plate 23 or the transverse plane 82 and a second cutting edge plane 40 can be an angle from 0-45 degrees. The backing plate 23 can also be at an angle to the transverse plane 82. Any one of the cutting planes formed by any of the leading cutting edges 18 could angle in an opposite direction from the angles illustrated. For example, the angle A between the transverse plane 82 and first cutting edge plane 39 narrows as it extends from the right side 77 to left side 78 of cutting head 20. Any of the cutting planes could be configured to narrow as it extends from the left side 78 to the right side 77 of the cutting head 20. Any of the cutting planes can be configured to angle in any direction, such as transverse to the right to left direction illustrated.

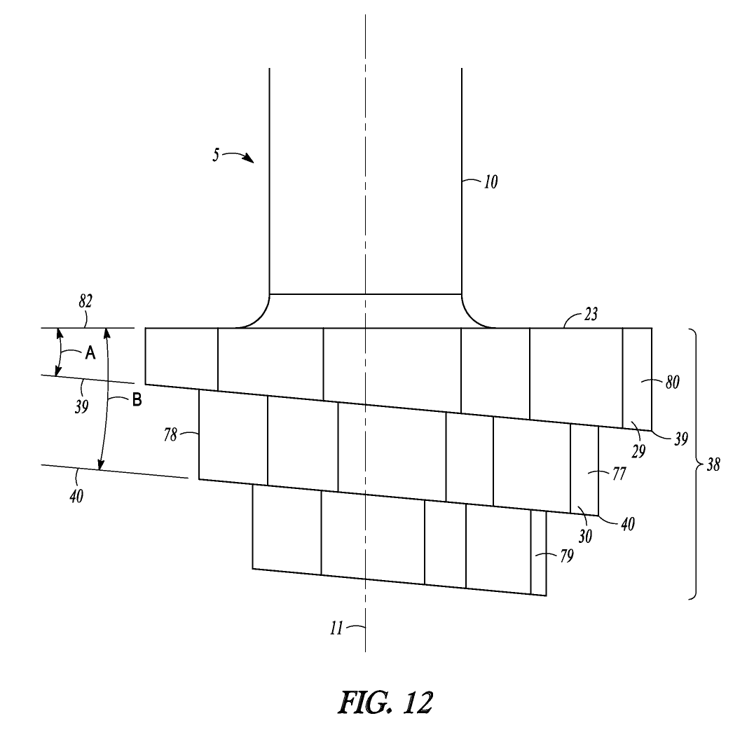

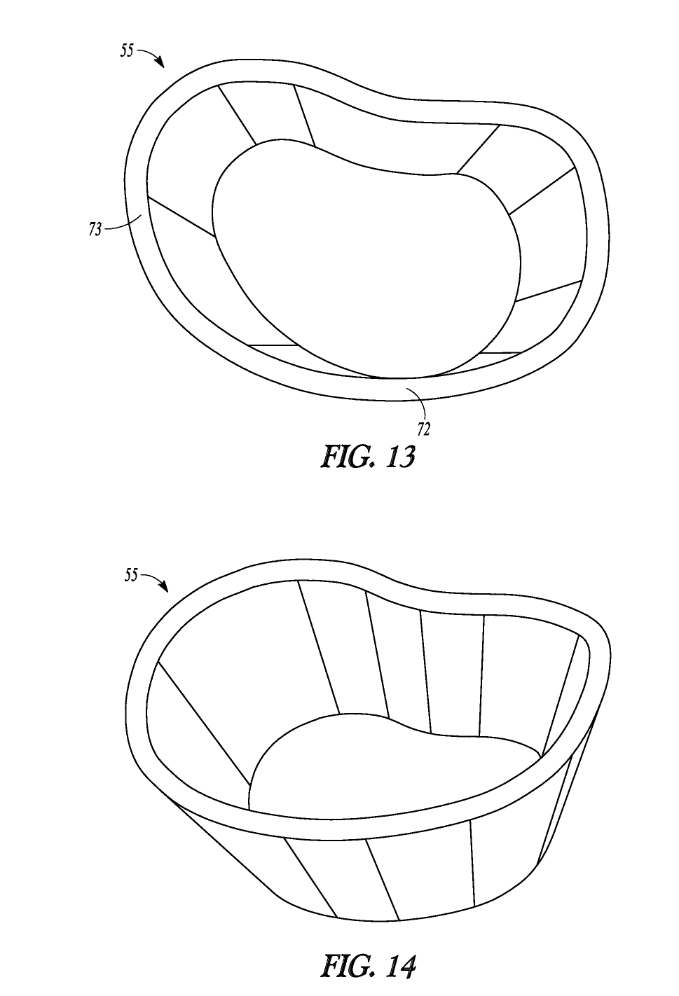

FIGS. 1-3 and 5-12 illustrate cutting heads 20 having cutting blade members 24 that are generally parallel to the long axis 11 of the handle 10. The cutting blade members 24 can be configured at angles to the long axis 11. FIG. 13 illustrates a conically shaped cutting blade member 55. Although this blade is shown as continuous, it can be configured as a non-continuous blade member such as the fourth annular blade member 28 in FIG. 3. Returning to FIG. 13, the conically shaped cutting blade member 55 can have a uniform taper, or can have portions that are at varied angular relationships to the longitudinal axis of the handle (See FIGS. 1-3). A small angle portion 72 can have an angle that is generally parallel to the longitudinal axis 11 of a punch 5 (see FIG. 12) or differing very slightly from the longitudinal axis 11. A large angle portion 73 can have an angle that is 0-45 degrees offset from the longitudinal axis 11. FIG. 14 illustrates the conically shaped cutting blade member 55 from a side viewpoint.

The above detailed description includes references to the accompanying drawings, which form a part of the detailed description. The drawings show, by way of illustration, specific embodiments in which the disclosure can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the disclosure should be determined with reference to the appended claims, along with the full scope of equivalents to which such claims are entitled.

1. A punch for cutting tissue in a resected proximal tibia, comprising:
an elongate handle having a longitudinal axis and including a proximal end and a distal end; and
a cutting head disposed at the distal end of the elongate handle, wherein said cutting head includes a first annular blade member with a first leading cutting edge that extends generally transversely to the longitudinal axis of the elongate handle, and wherein said cutting head includes a second annular blade member that is situated inwardly of said first annular blade member and which includes a second leading cutting edge that extends generally transversely to the longitudinal axis of the elongate handle.

2. The punch of claim 1, wherein at least one of the first leading cutting edge and the second leading cutting edge extends in multiple planes.

3. The punch of claim 1, wherein the first leading cutting edge extends in a first plane.

4. The punch of claim 3, wherein the second leading cutting edge extends in a second plane that is parallel with said first plane.

5. The punch of claim 3, wherein the second leading cutting edge extends in a second plane that is non-parallel with said first plane.

6. The punch of claim 1, wherein said second leading cutting edge extends distally of said first leading cutting edge.

7. The punch of claim 1, wherein the first leading cutting edge and the second leading cutting edge have the same perimeter shape.

8. The punch of claim 1, wherein at least one of the first leading cutting edge and the second leading cutting edge is curvilinear.

9. The punch of claim 1, wherein at least one of the first annular blade member and the second annular blade member includes a cylindrical longitudinal section.

10. The punch of claim 1, wherein at least one of the first annular blade member and the second annular blade member includes a conical longitudinal section.

11. The punch of claim 1, further comprising a backing plate from which at least one of the first annular blade member and the second annular blade member extends.

12. The punch of claim 1, wherein said cutting head further includes a first linear blade member that is surrounded by said second annular blade member and which includes a first linear cutting edge that extends generally transversely to the longitudinal axis of the elongate handle.

13. A punch for cutting tissue in a resected proximal tibia, comprising:
an elongate handle that includes a proximal end and a distal end; and
a cutting head disposed at the distal end of the elongate handle, wherein said cutting head includes a first annular blade member into which cut tissue surrounded by the first annular blade member can pass when the punch is impacted and a second annular blade member that is generally coaxial with said first annular blade member and into which cut tissue surrounded by the second annular blade member can pass when the punch is impacted into the resected proximal tibia, said first annular blade member including a first leading cutting edge, and said second annular blade member including a second leading cutting edge that encompasses a smaller area than said first leading cutting edge and that extends distally of said first leading cutting edge.

14. The punch of claim 13, wherein the first annular blade member and the second annular blade member are modular components separately or concurrently connectable to said elongate handle.

15. The punch of claim 13, wherein at least one of the first annular blade member and the second annular blade member extends in perpendicular manner from a backing plate situated at the distal end of the elongate handle.

16. A punch for cutting tissue in a resected proximal tibia, comprising:
an elongate handle that includes a proximal end and a distal end; and
a cutting head disposed at the distal end of the elongate handle, wherein said cutting head includes a first annular cutting edge for making a first circumferential cut in the resected proximal tibia, and wherein said cutting head includes a second annular cutting edge for making a second circumferential cut in the resected proximal tibia that is encompassed by said first circumferential cut.

17. The punch of claim 16, wherein the entirety of said second annular cutting edge extends further from said elongate handle than the entirety of said first annular cutting edge for cutting deeper into the resected proximal tibia than said first annular cutting edge.

18. The punch of claim 16, wherein at least one of said first circumferential cut and said second circumferential cut are coaxial.

19. The punch of claim 16, wherein the first annular cutting edge extends in a first plane, and wherein said second annular cutting edge extends in a second plane that is parallel with said first plane.

20. A method for cutting tissue in a resected proximal tibia, comprising:
providing a tibia punch that includes an outer annular blade member that is generally coaxial with an inner annular blade member, said inner annular blade member encompassing a smaller cutting area than said outer annular blade member and extending distally of said outer annular blade member; and
forcing the tibia punch into a resected proximal tibia, wherein the inner annular blade member enters the resected proximal tibia before the outer annular blade member, with an inner cut tissue surrounded by the inner annular blade member passing into the inner annular blade member and an outer cut tissue surrounded by the outer annular blade member passing into the outer annular blade member.

21. The method of claim 20, further comprising removing the tibia punch from the resected proximal tibia, wherein the inner cut tissue passes back through the inner annular blade member and the outer cut tissue passes back through the outer annular blade member, with the inner cut tissue and the outer cut tissue each including tissue portions still attached to the resected proximal tibia.

22. The method of claim 21, further comprising removing the inner cut tissue and the outer cut tissue from the resected proximal tibia which includes detaching the tissue portions from the resected proximal tibia.
